(19) United States
(12) Patent Application Publication
(10) Pub. No.: US 2012/0177736 A1
(43) Pub. Date: Jul. 12, 2012

(54) METHOD FOR TREATING A PATIENT IN NEED OF ASPIRIN THERAPY

(75) Inventor: JOHN R. PLACHETKA, Chapel Hill, NC (US)

(73) Assignee: POZEN INC., Chapel Hill, NC (US)

(21) Appl. No.: 13/345,075

(22) Filed: Jan. 6, 2012

Related U.S. Application Data
(63) Continuation of application No. 12/823,082, filed on Jun. 24, 2010.
(60) Provisional application No. 61/220,483, filed on Jun. 25, 2009.
(60) Provisional application No. 61/248,755, filed on Oct. 5, 2009.

(51) Int. Cl. A61K 9/28; A61K 9/00; A61P 29/00; A61P 1/14; A61K 31/616; A61P 1/04

(52) U.S. Cl. 424/472; 514/161; 424/400

(57) ABSTRACT

The present disclosure is directed to a method for treating a disease or disorder in a patient at risk of developing an NSAID-associated ulcer by administering to the patient in need thereof a pharmaceutical composition in unit dosage form comprising aspirin, or a pharmaceutically acceptable salt thereof, and an acid inhibitor to the at risk patient and thereby decreasing the patient's risk of developing an ulcer.

### CROSS REFERENCE TO RELATED APPLICATIONS

The present application claims the benefit of U.S. provisional application 61/220,483, filed on Jun. 25, 2009 and of U.S. provisional application 61/248,755, filed on Oct. 5, 2009, both of which are incorporated herein by reference in their entirety.

### FIELD OF THE INVENTION

The present disclosure is directed to a method for treating a disease or disorder in a patient at risk of developing a non-steroidal anti inflammatory drug (“NSAID”) -associated ulcer by administering to the patient in need thereof a pharmaceutical composition in unit dosage form comprising aspirin, or a pharmaceutically acceptable salt thereof, and an acid inhibitor to the at risk patient and thereby decreasing the patient's risk of developing an ulcer.

### BACKGROUND OF THE INVENTION

Aspirin is an NSAID, and is the general name for acetylsalicylic acid. Aspirin is used to reduce fever and provide pain relief from conditions such as muscle aches, toothaches, common colds, and headaches. It may also be used to reduce pain and inflammation in conditions such as arthritis, rheumatoid arthritis, ankylosing spondylitis, and osteoarthritis. Aspirin is also an anti-coagulant, and low-dose aspirin is often used to reduce blood clots that may lead to cardiovascular disease, including heart attack and stroke. In addition to its preventative use, it is also used in treatment of cardiovascular diseases. Low-dose aspirin is recommended for the prevention of cardiovascular and cerebrovascular events, and an estimated 50 million people in the United States take aspirin for cardioprotection.

Aspirin is a potent inhibitor of thromboxane A2 (“TxA2”) synthesis by platelets, reducing their aggregation and adhesion and thus reducing the risk of arterial thrombosis (Awtry, et al., Circulation 101:1206-1218 (2000); Gengo, et al., J. Clin. Pharmacol. 48:335-343 (2008)). This cardioprotective benefit of aspirin is not realized with antiplatelet drugs until platelet TxA2 generation is reduced by more than 95% in serum (Patrono, et al., New Eng. J. Med. 353:2373-2382 (2005); Grosser, et al. “Thromboxane Generation,” in Platelets, 2nd ed., Michelson ed., Elseiver (2007)). For example, 95% inhibition of TxA2 in plasma corresponds to only a 50-75% reduction in the urinary excretion rates of the TxA2 metabolite, 11-dh-TXB₂, due to the extraplatelet sources of this urinary metabolite (Hart, et al., Pharmacotherapy 23(5):579-584 (2003)).

While aspirin and other NSAIDs remain a key therapy for pain, inflammation, and cardiovascular disease, there is a substantial risk of upper gastrointestinal (“UGI”) ulcerations and ulcer complications, such as, for example, bleedings and perforations, with chronic NSAID therapy. This risk increases with use over time. The cumulative incidence of gastroduodenal ulcers (“GDUs”) with conventional NSAID use has been reported to be as high as 25-30% at 3 months and 45% at 6 months versus 3-7% for placebo. At any given time, the incidence of UGI ulcers in NSAID users has been estimated to be as high as 30%. Certain risk factors associated with an NSAID user developing UGI ulcers are: age ≧50 years and history of UGI ulcer or bleeding. The mechanism associated with the increased incidence of ulcers in chronic NSAID users may be complex, but it is thought that gastric acid, combined with a reduction in protective mechanisms of the UGI mucosa, contribute to this pathology. UGI mucosal injury includes petechia, erosions and ulcers. In addition, once mucosal injury occurs, acid has the ability to impair normal hemostasis and healing. These factors, coupled with the known anti-platelet effect of some NSAIDs, may increase the risk for gastrointestinal (“GI”) injury and bleeding. UGI effects of NSAIDs also include: dyspepsia (experienced by up to 40% of patients on NSAID therapy), erosive esophagitis (“EE”) (experienced by 21% of regular NSAID users), and an increase in gastroesophageal reflux disease symptoms.

Because of these risks, physicians generally prefer to prescribe low-dose aspirin for preventing cardiovascular disease or stroke, even though low-dose aspirin does not have the same beneficial therapeutic effects as high-dose aspirin. Instead, physicians generally only prescribe high-dose aspirin if the therapeutic benefits outweigh the risks associated with aspirin therapy, for example if the patient has existing cardiovascular disease. Thus, if a formulation of aspirin reduces the risks associated with aspirin therapy, it would be preferable to have patients on high-dose aspirin therapy, for example for preventative treatment of cardiovascular disease or stroke. Thus, there is a need in the art for a formulation of aspirin that reduces the risks associated with aspirin therapy, particularly during chronic treatment.

### SUMMARY OF THE INVENTION

The present disclosure is based upon the discovery of an aspirin combination treatment that reduces the risks associated with aspirin therapy, for example undesirable gastrointestinal side effects and other safety concerns, particularly during chronic treatment. In certain embodiments, the treatment involves the administration of a single, coordinated, unit dosage form that combines: a) an acid inhibitor that raises intragastric pH levels; and b) aspirin that is specially formulated to be released in a coordinated way with the acid inhibitor, such that administration of the unit dosage form reduces the risks associated with aspirin therapy, for example any adverse effects the aspirin may have on gastroduodenal mucosa. Either short- or long-acting acid inhibitors can be effectively used in the unit dosage forms disclosed herein. In certain embodiments, this treatment has the added benefit of being able to protect patients from other gastrointestinal ulcerogens whose effect may otherwise be enhanced by the disruption of gastroprotective prostaglandins due to aspirin therapy.

In one aspect, the disclosure is directed to preventing or treating a disease or disorder in a patient at risk of developing an NSAID-associated ulcer by administration of the pharmaceutical compositions in unit dosage form disclosed herein. In another embodiment, administration of the pharmaceutical compositions in unit dosage form disclosed herein to treat a disease or disorder in a patient at risk of developing an NSAID-associated ulcer decreases the risk of the patient developing an ulcer, including but not limited to decreasing the risk of the occurrence of a gastroduodenal ulcer or a duodenal ulcer. In yet another embodiment, administration of the pharmaceutical compositions in unit dosage form disclosed herein to treat a disease or disorder in a patient at risk of developing an NSAID-associated ulcer reduces the patient's heartburn associated symptoms. In still another embodiment, administration of the pharmaceutical compositions in unit dosage form disclosed herein to treat a disease or disorder in a patient at risk of developing an NSAID-associated ulcer reduces the patient's dyspepsia associated symptoms. In yet another embodiment, administration of the pharmaceutical compositions in unit dosage form disclosed herein to treat a disease or disorder in a patient at risk of developing an NSAID-associated ulcer reduces the patient's level of urinary 11-dehydrothromboxane. In another aspect, the disclosure is directed to preventing or treating a disease or disorder in a patient in need thereof wherein the disease or disorder is pain, inflammation, arthritis osteoarthritis, rheumatoid arthritis, ankylosing spondylitis, headache, toothache, common cold, muscle ache, cardiovascular disease, cancer, cerebrovascular disease, or combinations thereof

In each of the embodiments disclosed herein the pharmaceutical composition in unit dosage form administered to the patient comprises: a) a therapeutically effective amount of an acid inhibitor in an amount sufficient to raise the gastric pH of the patient to at least about 3.5, 4.0, 4.5, 5.0, 5.5, or higher upon administration of one or more of the unit dosage forms, and b) a therapeutically effective amount of aspirin, or a pharmaceutically acceptable salt thereof; wherein the aspirin, or a pharmaceutically acceptable salt thereof, is released from the unit dosage form only when the pH of the surrounding medium or environment is about 3.5, 4.0, 4.5, 5.0, 5.5 or higher. In some embodiments, the pharmaceutical composition in unit dosage form comprises a) a therapeutically effective amount of an acid inhibitor that is immediately soluble when the dosage form is place in an aqueous medium, independent of pH, for example in an amount effective to raise the gastric pH of the patient to at least about 3.5, 4.0, 4.5, 5.0, 5.5, or higher upon administration of one or more of the unit dosage forms. In other embodiments, the pharmaceutical composition in unit dosage form comprises a) an acid inhibitor in an amount effective to raise the gastric pH of the patient to at least 3.5, 4.0, 4.5, 5.0, 5.5, or higher upon administration of one or more of the unit dosage forms. In certain embodiments, the pharmaceutical composition in unit dosage form comprises b) a therapeutically effective amount of aspirin, or a pharmaceutically acceptable salt thereof; wherein the aspirin or a pharmaceutically acceptable salt thereof is surrounded by a coating that is substantially insoluble in an aqueous medium at a pH below about 3.5, 3.0, 2.5, 2.0, 1.5, or lower. In other embodiments, the pharmaceutical composition in unit dosage form comprises b) aspirin or a pharmaceutically acceptable salt thereof, wherein the aspirin or a pharmaceutically acceptable salt thereof is released from the unit dosage form only when the pH of the surrounding medium or environment is about 3.5, 4.0, 4.5, 5.0, 5.5, or higher. In certain embodiments, the aqueous medium is also at a temperature of about 37° C.

In still other embodiments, a therapeutically effective amount of an acid inhibitor is an amount sufficient to raise the gastric pH of the patient to at least about 3.5, 4.0, 4.5, 5.0, 5.5, or higher upon administration of one or more of the unit dosage forms wherein the unit dosage form provides for coordinated release of the acid inhibitor and the aspirin such that: i) at least a portion of the acid inhibitor is released independent of the pH of the surrounding medium or environment; and ii) the aspirin, or a pharmaceutically acceptable salt thereof, is not released from the unit dosage form until the pH of the surrounding medium is 3.5, 4.0, 4.5, 5.0, 5.5, or higher. Such pharmaceutical compositions have been described in U.S. Pat. No. 6,926,907, which is incorporated herein by reference in its entirety. In still other embodiments, the pharmaceutical composition in unit dosage form comprises any mixture of the above described acid inhibitor and aspirin, or a pharmaceutically acceptable salt thereof.

In certain embodiments of the present disclosure, the risk of NSAID-associated gastrointestinal ulcer in a patient may be associated with short-term or chronic NSAID treatment, age of the patient (for example if the patient is 50 years of age or older), or a combination thereof. In the embodiments disclosed herein, a pharmaceutical composition in unit dosage form is administered to the patient for 7 days, 10 days, 14 days, 15 days, 16 days, 17 days, 18 days, 19 days, 20 days, 21 days, 22 days, 23 days, 24 days, 25 days, 26 days, 27 days, 28 days, 29 days, 1 month, 2 months, 3 months, 4 months, 5 months, 6 months, 12 months, 18 months, or longer. In other embodiments, a pharmaceutical composition in unit dosage form is administered to the patient frequently or chronically.

In another aspect, the pharmaceutical composition in unit dose form disclosed herein decreases the risk of the patient developing a gastric ulcer, duodenal ulcer, or both. In yet another aspect, the disease or disorder treated by the pharmaceutical compositions disclosed herein include but are not limited to pain, inflammation, arthritis, osteoarthritis, rheumatoid arthritis, ankylosing spondylitis, headache, toothache, common cold, muscle ache, cardiovascular disease, cancer (e.g., colon cancer) or any combination thereof. In other embodiments, the pharmaceutical composition in unit dose form disclosed herein may be administered to prevent or treat cardiovascular disease or cerebrovascular disease.

Numerous studies have provided evidence that NSAIDs, including aspirin, may prevent cancer. Experimental and epidemiologic (nonrandomized) studies, along with randomized clinical trials, have shown that NSAIDs may have a prophylactic effect against certain cancers. These results have been confirmed in certain colorectal cancers and suggested for other cancer sites. In other embodiments, the pharmaceutical composition in unit dose form disclosed herein may be administered to prevent or treat cancer, including but not limited to biliary tract cancer; brain cancer; breast cancer; cervical cancer; choriocarcinoma; colon cancer; endometrial cancer; esophageal cancer; fibrosarcoma, gastric cancer; hepatoma, intraepithelial neoplasms; lymphomas; liver cancer; lung cancer (e.g., small cell and non-small cell); melanoma; neuroblastomas; oral cancer; ovarian cancer; pancreatic cancer; prostate cancer; rectal cancer; sarcomas; skin cancer; testicular cancer; thyroid cancer; renal cancer, glioblastoma, adenocarcinoma, adenoma, astrocytoma, bladder tumor, bone carcinoma, brain carcinoma, Burkitt lymphoma, Kaposi Sarcoma, non-Hodgkins lymphoma, Hodgkins lymphoma, gastric tumor, breast carcinoma, cervical carcinoma, colon carcinoma, kidney carcinoma, liver carcinoma, lung carcinoma, ovarian carcinoma, pancreatic carcinoma, prostate carcinoma, rectal carcinoma, skin carcinoma, stomach carcinoma, testis carcinoma, thyroid carcinoma, chondrosarcoma, choriocarcinoma, fibroma, fibrosarcoma, glioblastoma, glioma, hepatoma, histiocytoma, leiomyoblastoma, leiomyosarcoma, leukemia, lymphoma, liposarcoma cell, mammary carcinoma, medulloblastoma, melanoma, metastases, muscle tumor, myeloma, ovarian carcinoma, plasmacytoma, neuroblastoma, neuroglioma, osteogenic sarcoma, pancreatic tumor, pituitary carcinoma, renal tumors, retinoblastoma, rhabdomyosarcoma, sarcoma, testicular tumor, thymoma, uterine carcinoma, Wilms' tumor, as well as other carcinomas and sarcomas. In particular embodiments, the pharmaceutical compositions disclosed herein are administered to a patient to prevent or treat colon cancer or colorectal cancer.

In a further aspect, the pharmaceutical compositions in unit dosage form disclosed herein may comprise an acid inhibitor in an amount effective to raise the pH of the gastric fluid of a patient to at least 3.5, at least 4.0, at least 4.5, at least 5.0, at least 5.5 or greater when the dosage form is administered to the patient, for example orally administered. The acid inhibitor may be present in the unit dosage form in an amount of from about 5 mg to about 1000 mg. In certain embodiments, the acid inhibitor is omeprazole, esomeprazole, lansoprazole, pantoprazole, rabeprazole, dexlansoprazole, and tenatoprazole, or pharmaceutically acceptable salts thereof. In particular embodiments, the pharmaceutical compositions in unit dosage forms disclosed herein comprise omeprazole, or a pharmaceutically acceptable salt thereof, in an amount of, for example, about 10 mg, 15 mg, 20 mg, 25 mg, 30 mg, 35 mg, 40 mg, 45 mg, 50 mg, 60 mg, 70 mg, 80 mg, 90 mg, or 100 mg. In other embodiments, the pharmaceutical compositions in unit dosage forms disclosed herein comprise aspirin, or a pharmaceutically acceptable salt thereof, in an amount of, for example, from about 30 mg to about 1300 mg, or at an amount of about 75 mg, 81 mg, 100 mg, 150 mg, 162 mg, 300 mg, 325 mg, 500 mg, or 650 mg.

In a still further aspect, the pharmaceutical composition is formulated for administration to a patient one or more times daily. In one embodiment, the unit dosage form is suitable for oral administration. In certain embodiments, the unit dosage form may be a tablet, a sequential-delivery tablet formulation, a capsule, a capsule containing beads or minitablets. In one aspect, the unit dosage form is a tablet comprising a core and two or more layers, in which i) the core comprises aspirin or a pharmaceutically acceptable salt thereof; ii) a first layer surrounds the core and the layer is a coating that is substantially insoluble in aqueous medium at a pH below 3.5, for example below 3.0, 2.5, 2.0, 1.5, 1.0, or lower and/or at a temperature of about 37° C.; and iii) at least one second layer surrounds the first layer and comprises the acid inhibitor. In some embodiments, the first layer may be, for example, an enteric coating (“EC”) or a time-release coating. In other embodiments, the unit dosage form may be further surrounded by a pharmacologically inert, water soluble coating or film. In another embodiment, the administration of the unit dosage form disclosed herein improves compliance in a patient who requires short-term or chronic daily dosages of aspirin or a pharmaceutically acceptable salt thereof.

In one aspect, administering a pharmaceutical composition in unit dosage form to a patient is more effective at decreasing the risk of developing an ulcer than treatment with only aspirin, for example enteric-coated or non-enteric-coated aspirin, or a pharmaceutically acceptable salt thereof. In another aspect, administering a pharmaceutical composition in unit dosage form disclosed herein to a patient reduces the patient's heartburn associated symptoms more than treating the patient in need thereof with only aspirin, for example enteric coated or non-enteric coated aspirin, or a pharmaceutically acceptable salt thereof. In still another aspect, administering a pharmaceutical composition in unit dosage form disclosed herein to a patient reduces the patient's dyspepsia more than treating the patient in need thereof with only aspirin, for example enteric coated or non-enteric coated aspirin, or a pharmaceutically acceptable salt thereof. In yet another aspect, administering a pharmaceutical composition in unit dosage form disclosed herein to a patient reduces the patient's level of urinary 11-dehydrothromboxane more than treating the patient in need thereof with only aspirin, for example enteric coated or non-enteric coated aspirin, or a pharmaceutically acceptable salt thereof.

Another embodiment of the present disclosure is a solid pharmaceutical composition in unit dosage form suitable for oral administration to a mammal, comprising: a) omeprazole or pharmaceutically acceptable salt thereof that is immediately soluble when the dosage form is placed in an aqueous medium, independent of pH; and b) aspirin or a pharmaceutically acceptable salt thereof, surrounded by a coating that is substantially insoluble in an aqueous medium at a pH below 3.5 and/or at a temperature of about 37° C. In one embodiment, the omeprazole or pharmaceutically acceptable salt thereof is present in the composition in an amount effective to raise the pH of the gastric fluid of a mammal to at least about 3.5, 4.0, 4.5, 5.0, 5.5 or higher when the dosage form is administered orally to the mammal. In another embodiment, the amount of aspirin, or a pharmaceutically acceptable salt thereof, is about 75 mg, 81 mg, 100 mg, 150 mg, 162 mg, 300 mg, 325 mg, 500 mg, or 650 mg. In yet another embodiment, the amount of omeprazole, or a pharmaceutically acceptable salt thereof, is about 10 mg, 15 mg, 20 mg, 25 mg, 30 mg, 35 mg, 40 mg, 45 mg, 50 mg, 60 mg, 70 mg, 80 mg, 90 mg, or 100 mg. The solid pharmaceutical composition in unit dosage form may be formulated to be administered to a patient one or more times daily. In certain embodiments, the solid pharmaceutical composition in unit dosage form is suitable for oral administration. In other embodiments, the solid pharmaceutical composition in unit dosage form may be a tablet, a sequential-delivery tablet formulation, a capsule, a capsule containing beads or minitablets. In another aspect, the solid pharmaceutical composition in unit dosage form is a tablet comprising a core and two or more layers, in which i) the core comprises aspirin or a pharmaceutically acceptable salt thereof; ii) a first layer surrounds the core and the layer is a coating that is substantially insoluble in aqueous medium at a pH below 3.5, for example below 3.0, 2.5, 2.0, 1.5, 1.0, or lower and/or at a temperature of about 37° C.; and iii) at least one second layer surrounds the first layer and comprises omeprazole or pharmaceutically acceptable salt. In some embodiments, the first layer may be, for example, an enteric coating (“EC”) or a time-release coating. In other embodiments, the solid pharmaceutical composition in unit dosage form may be further surrounded by a pharmacologically inert, water soluble coating or film.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates pooled gastroduodenal data from three Phase I studies on PA32520 and PA32540. Further information regarding FIG. 1 may be found below in Example 1.

FIG. 2 illustrates the change in urinary 11-dh-TXB₂ at Day 28 in a Phase I study on PA32520. Further information regarding FIG. 2 may be found below in Example 2.

FIG. 3 shows a release profile of PA32540 and is described more fully below in Example 3.

### DETAILED DESCRIPTION OF THE INVENTION

The term “acid inhibitor” includes without limitation proton pump inhibitors and histamine H₂ receptor antagonists. Examples of proton pump inhibitors include but are not limited to omeprazole, esomeprazole, lansoprazole, pantoprazole, rabeprazole, dexlansoprazole, and tenatoprazole. Examples of histamine H₂ receptor antagonists include but are not limited to cimetidine, ranitidine, ebrotidine, pabutidine, lafutidine, loxtidine, nizatidine, and famotidine.

The term “at risk patient” refers to patient(s) at risk for NSAID associated ulcer due to age ≧50 years, or a patient who has a history of UGI ulcer or bleeding. In one embodiment, the at risk patient is a patient at risk for NSAID associated ulcer due to age greater than or equal to 50 years. In yet another embodiment, the at risk patient is a patient at risk for NSAID associated ulcer due to history of UGI ulcer or bleeding.

The term “enantiomerically pure” refers to a compound containing at least about 75% of the named enantiomer out of the total amount of the two possible enantiomers contained therein. In various embodiments, “enantiomerically pure” refers to a compound containing at least about 90%, about 95%, about 96%, about 97%, about 98%, about 99%, or about 99.9% of the named enantiomer out of the total amount of the two possible enantiomers contained therein.

The term “pharmaceutically acceptable,” as employed herein, indicates the subject matter being identified as “pharmaceutically acceptable” is suitable and physiologically acceptable for administration to a patient/subject. For example, the term “pharmaceutically acceptable salt(s)” denotes suitable and physiologically acceptable salt(s).

The phrase “aspirin or pharmaceutically acceptable salt thereof” refers to the free base of aspirin, pharmaceutically acceptable salt(s) of aspirin, and/or mixtures of the free base of aspirin and at least one pharmaceutically acceptable salt of aspirin.

The phrase “omeprazole, or pharmaceutically acceptable salt thereof” refers to the free base of omeprazole, pharmaceutically acceptable salt(s) of omeprazole, and/or mixtures of the free base of omeprazole and at least one pharmaceutically acceptable salt of omeprazole.

The term “unit dosage form” or “unit dose form” as used herein refers to a single entity for drug administration. For example, a single tablet or capsule containing both an acid inhibitor and aspirin or a pharmaceutically acceptable salt thereof is a unit dosage form. Unit dosage forms of the present disclosure can provide for sequential drug release in a way that elevates gastric pH and reduces the deleterious effects of aspirin on the gastroduodenal mucosa, e.g., the acid inhibitor is released first and the release of aspirin is delayed until after the pH in the GI tract has risen to at least 3.5, 4.0, 4.5, 5.0, 5.5, or greater. A “unit dosage form” may also be referred to as a “fixed dosage form” or a “fixed dosage combination” and are otherwise interchangeable.

With regard to the dosages of aspirin or a pharmaceutically acceptable salt thereof and/or an acid inhibitor, the term “about” is intended to reflect variations from the specifically identified dosages that are acceptable within the art. With regard to the pH values and/or ranges recited herein, the term “about” is intended to capture variations above and below the stated number that may achieve substantially the same results as the stated number.

With regard to the term numerical values used in conjunction with the phrase “substantially free,” the term is intended to capture variations above and below the stated number that may achieve substantially the same results as the stated number. The phrase “substantially free” means from about 95% to about 99.99% free. For example, substantially free may mean about 95% free, about 96% free, about 97% free, about 98% free, about 99% free, or about 99.99% free. In the present disclosure, each of the variously stated ranges is intended to be continuous so as to include each numerical parameter between the stated minimum and maximum value of each range. For example, a range of about 1 to about 4 includes about 1, 1, about 2, 2, about 3, 3, about 4, and 4.

One embodiment is directed to a method comprising: treating a disease or disorder in a patient at risk of developing an NSAID-associated ulcer by administering to the patient in need thereof a pharmaceutical composition in unit dosage form comprising a) an acid inhibitor in an amount sufficient to raise the gastric pH of the patient to at least about 3.5, 4.0, 4.5, 5.0, 5.5, or greater upon administration of one or more of the unit dosage forms, and b) a therapeutically effective amount of aspirin, or a pharmaceutically acceptable salt thereof; wherein the unit dosage form provides for coordinated release of the acid inhibitor and the aspirin such that: i) at least a portion of the acid inhibitor is released independent of the pH of the surrounding medium; and ii) the aspirin, or a pharmaceutically acceptable salt thereof, is not released from the unit dosage form until the pH of the surrounding medium is at least about 3.5, 4.0, 4.5, 5.0, 5.5, or higher; and wherein the pharmaceutical composition in unit dosage form decreases the risk of the patient developing an ulcer.

Another embodiment is directed to a method comprising: treating a disease or disorder in a patient in need of chronic NSAID treatment and at risk of developing an NSAID-associated ulcer by administering to the patient in need thereof a pharmaceutical composition in unit dosage form comprising a) an acid inhibitor in an amount sufficient to raise the gastric pH of the patient to at least about 3.5, 4.0, 4.5, 5.0, 5.5 or higher upon administration of one or more of the unit dosage forms, and b) a therapeutically effective amount of aspirin, or a pharmaceutically acceptable salt thereof; wherein the unit dosage form provides for coordinated release of the acid inhibitor and the aspirin such that: i) at least a portion of the acid inhibitor is released independent of the pH of the surrounding medium; and ii) the aspirin, or a pharmaceutically acceptable salt thereof, is not released from the unit dosage form until the pH of the surrounding medium is at least about 3.5, 4.0, 4.5, 5.0, 5.5 or higher; and wherein the pharmaceutical composition in unit dosage form decreases the risk of the patient developing an ulcer.

Still another embodiment is directed to a method comprising: treating signs and symptoms of pain, inflammation, osteoarthritis, rheumatoid arthritis, ankylosing spondylitis, headache, toothache, common cold, muscle ache, cardiovascular disease, cancer, or any combination thereof in a patient at risk of developing an NSAID-associated ulcer by administering to the patient in need thereof a pharmaceutical composition in unit dosage form comprising a) an acid inhibitor in an amount sufficient to raise the gastric pH of the patient to at least about 3.5, 4.0, 4.5, 5.0, 5.5 or higher upon administration of one or more of the unit dosage forms, and b) a therapeutically effective amount of aspirin, or a pharmaceutically acceptable salt thereof; wherein the unit dosage form provides for coordinated release of the acid inhibitor and the aspirin such that: i) at least a portion of the acid inhibitor is released independent of the pH of the surrounding medium; and ii) the aspirin, or a pharmaceutically acceptable salt thereof, is not released from the unit dosage form until the pH of the surrounding medium is at least about 3.5, 4.0, 4.5, 5.0, 5.5 or higher; and wherein the pharmaceutical composition in unit dosage form decreases the risk of the patient developing an ulcer.

Still yet another embodiment is directed to a method comprising: treating signs and symptoms of pain, inflammation, osteoarthritis, rheumatoid arthritis, ankylosing spondylitis, headache, toothache, common cold, muscle ache, cardiovascular disease, cancer, or any combination thereof in a patient in need of chronic NSAID treatment and at risk of developing an NSAID-associated ulcer by administering to the patient in need thereof a pharmaceutical composition in unit dosage form comprising a) an acid inhibitor in an amount sufficient to raise the gastric pH of the patient to at least about 3.5, 4.0, 4.5, 5.0, 5.5 or higher upon administration of one or more of the unit dosage forms, and b) a therapeutically effective amount of aspirin, or a pharmaceutically acceptable salt thereof; wherein the unit dosage form provides for coordinated release of the acid inhibitor and the aspirin such that: i) at least a portion of the acid inhibitor is released independent of the pH of the surrounding medium; and ii) the aspirin, or a pharmaceutically acceptable salt thereof, is not released from the unit dosage form until the pH of the surrounding medium is at least about 3.5, 4.0, 4.5, 5.0, 5.5 or higher; and wherein the pharmaceutical composition in unit dosage form decreases the risk of the patient developing an ulcer.

In a further embodiment, the disease or disorder treated by the pharmaceutical compositions disclosed herein is selected from pain and inflammation. In yet another embodiment, the disease or disorder treated by the pharmaceutical compositions disclosed herein is osteoarthritis, rheumatoid arthritis, or ankylosing spondylitis. A still another embodiment, the disease or disorder treated by the pharmaceutical compositions disclosed herein is headache, toothache, common cold, muscle ache, cardiovascular disease, or any combination thereof. In another embodiment, the disease or disorder treated by the pharmaceutical compositions disclosed herein is cancer. In yet a further embodiment, the patient at risk of developing an NSAID associated ulcer is ≧50 years old. In still yet another embodiment, the patient at risk of developing an NSAID associated ulcer has a history of UGI ulcer or bleeding.

In a further embodiment, the pharmaceutical composition in unit dosage form decreases the risk of the patient developing a gastroduodenal ulcer. In yet a further embodiment, the pharmaceutical composition in unit dosage form decreases the risk of the patient developing a duodenal ulcer. In a further embodiment, the pharmaceutical composition in unit dosage form decreases the risk of the patient developing a gastric ulcer.

In another embodiment, administering the pharmaceutical composition in unit dosage form of the present disclosure to patients in need of NSAID treatment results in fewer patients developing a gastric ulcer than patients in need of NSAID treatment who are administered aspirin, whether enteric coated or non-enteric coated aspirin. In yet another embodiment, administering the pharmaceutical composition in unit dosage form of the present disclosure to patients in need of NSAID treatment results in fewer patients developing a duodenal ulcer than patients in need of NSAID treatment who are administered aspirin, whether enteric coated or non-enteric coated aspirin. In still another embodiment, administering the pharmaceutical composition in unit dosage form of the present disclosure to patients in need of NSAID treatment results in fewer patients developing heartburn associated symptoms than patients in need of NSAID treatment who are administered aspirin, whether enteric coated or non-enteric coated aspirin. In another embodiment, administering the pharmaceutical composition in unit dosage form of the present disclosure to patients in need of NSAID treatment results in fewer patients developing dyspepsia than patients in need of NSAID treatment who are administered aspirin, whether enteric coated or non-enteric coated aspirin. In yet another embodiment, administering the pharmaceutical composition in unit dosage form of the present disclosure to patients in need of NSAID treatment reduces the patents' level of urinary 11-dehydrothromboxane compared to patients in need of NSAID treatment who are administered aspirin, whether enteric coated or non-enteric coated aspirin. In a yet still further embodiment, the patient is treated longer with the pharmaceutical composition in unit dosage form of the present disclosure than with aspirin, whether enteric coated or non-enteric coated aspirin. In yet another embodiment, patient compliance with long-term treatment is improved with the pharmaceutical compositions disclosed herein as compared to aspirin, whether enteric coated or non-enteric coated aspirin.

In a yet even further embodiment, the pharmaceutical composition in unit dosage form is a multilayer tablet comprising at least one core and at least a first layer and a second layer, wherein:
i) the core comprises aspirin, or a pharmaceutically acceptable salt thereof;
ii) the first layer is a coating that at least begins to release the aspirin, or a pharmaceutically acceptable salt thereof, when the pH of the surrounding medium is about 3.5, 4.0, 4.5, 5.0, 5.5 or greater; and
iii) the second layer comprises an acid inhibitor, wherein at least some of the acid inhibitor is released at a pH of from about 0 or greater, for example 0.5, 1.0, 1.5, 2.0, 2.5, or 3.0.

In a further embodiment, the acid inhibitor is released from the multilayer tablet at a pH of from about 1.0 or greater. In a yet further embodiment, the acid inhibitor is released from the multilayer tablet at a pH of from about 0 to about 2.0. In a still further embodiment, at least a portion of the acid inhibitor contained in the multilayer tablet is not coated with an enteric coating. In a yet still further embodiment, the first layer of the multilayer tablet is an enteric coating or a time-release coating. In a yet even still further embodiment, the multi-layer tablet is substantially free of sodium bicarbonate. In a still further embodiment, the acid inhibitor is enantiomerically pure.

In another embodiment, the therapeutically effective amount of aspirin, or a pharmaceutically acceptable salt thereof, in the pharmaceutical compositions disclosed herein is selected from 30 mg and 1300 mg. In a still yet further embodiment, the therapeutically effective amount of aspirin, or a pharmaceutically acceptable salt thereof, is 81 mg. In a still yet further embodiment, the therapeutically effective amount of aspirin, or a pharmaceutically acceptable salt thereof, is 325 mg. In an even still further embodiment, the therapeutically effective amount of aspirin, or a pharmaceutically acceptable salt thereof, is 650 mg. In another embodiment, the therapeutically effective amount of aspirin, or a pharmaceutically acceptable salt thereof, is 75 mg, 100 mg, 150 mg, 162 mg, 300 mg, or 500 mg. In another embodiment, aspirin can be present as the free base. In yet another embodiment, aspirin can be present in equivalent amounts of pharmaceutically acceptable salts of aspirin.

In one embodiment, the pharmaceutical composition in unit dosage form comprises about 30-1300 mg of the aspirin, or a pharmaceutically acceptable salt thereof, and about 1-1000 mg of the acid inhibitor. In another embodiment, the pharmaceutical composition in unit dosage form comprises about 30-1300 mg of the aspirin, or a pharmaceutically acceptable salt thereof, and about 5-650 mg of a proton pump inhibitor. In another embodiment, the pharmaceutical composition in unit dosage form comprises about 30-1300 mg of the aspirin, or a pharmaceutically acceptable salt thereof, and about 5-50 mg omeprazole, or a pharmaceutically acceptable salt thereof, or about 15, 20, 30, or 40 mg omeprazole, or a pharmaceutically acceptable salt thereof. In yet another embodiment, the pharmaceutical composition in unit dosage form comprises about 30-1300 mg of the aspirin, or a pharmaceutically acceptable salt thereof, and about 5-100 mg esomeprazole, or a pharmaceutically acceptable salt thereof, or about 20, 30, or 40 mg esomeprazole, or a pharmaceutically acceptable salt thereof. In yet another embodiment, the pharmaceutical composition in unit dosage form comprises about 30-1300 mg of the aspirin, or a pharmaceutically acceptable salt thereof, and about 10-150 lansoprazole, or a pharmaceutically acceptable salt thereof. In still another embodiment, the pharmaceutical composition in unit dosage form comprises about 30-1300 mg of the aspirin, or a pharmaceutically acceptable salt thereof, and about 10-200 pantoprazole, or a pharmaceutically acceptable salt thereof. In another embodiment, the pharmaceutical composition in unit dosage form comprises about 30-1300 mg of the aspirin, or a pharmaceutically acceptable salt thereof, and about 15-100 mg dexlansoprazole, or a pharmaceutically acceptable salt thereof. In yet another embodiment, the pharmaceutical composition in unit dosage form comprises about 30-1300 mg of the aspirin, or a pharmaceutically acceptable salt thereof, and about 10-150 mg tenatoprazole, or a pharmaceutically acceptable salt thereof.In another embodiment, the pharmaceutical composition in unit dosage form comprises about 30-1300 mg of the aspirin, or a pharmaceutically acceptable salt thereof, and about 5-100 mg rabeprazole, or a pharmaceutically acceptable salt thereof, or about 20 mg rabeprazole, or a pharmaceutically acceptable salt thereof.

In one embodiment, the pharmaceutical composition in unit dosage form comprises about 81 mg of the aspirin, or a pharmaceutically acceptable salt thereof, and about 20 mg omeprazole, or a pharmaceutically acceptable salt thereof. In another embodiment, the pharmaceutical composition in unit dosage form comprises about 325 mg of the aspirin, or a pharmaceutically acceptable salt thereof, and about 20 mg omeprazole, or a pharmaceutically acceptable salt thereof. In still another embodiment, the pharmaceutical composition in unit dosage form comprises about 81 mg of the aspirin, or a pharmaceutically acceptable salt thereof, and about 40 mg omeprazole, or a pharmaceutically acceptable salt thereof. In yet another embodiment, the pharmaceutical composition in unit dosage form comprises about 325 mg of the aspirin, or a pharmaceutically acceptable salt thereof, and about 40 mg omeprazole, or a pharmaceutically acceptable salt thereof. In one embodiment, the pharmaceutical composition in unit dosage form comprises about 650 mg of the aspirin, or a pharmaceutically acceptable salt thereof, and about 15 mg omeprazole, or a pharmaceutically acceptable salt thereof. In another embodiment, the pharmaceutical composition in unit dosage form comprises about 650 mg of the aspirin, or a pharmaceutically acceptable salt thereof, and about 20 mg omeprazole, or a pharmaceutically acceptable salt thereof. In yet another embodiment, the pharmaceutical composition in unit dosage form comprises about 650 mg of the aspirin, or a pharmaceutically acceptable salt thereof, and about 40 mg omeprazole, or a pharmaceutically acceptable salt thereof.

In certain embodiments, the duration of treatment may be approximately 1 week, 10 days, 2 weeks, 1 month, 2 months, 3 months, 4 months, 5 months, 6 months or longer, and may be chronic treatment.

In an even further embodiment, the pharmaceutical composition in unit dosage form is a multilayer tablet comprising a core comprising aspirin, or a pharmaceutically acceptable salt thereof, and a first layer comprising a coating that at least begins releasing the aspirin when the pH of the surrounding medium is about 3.5, 4.0, 4.5, 5.0, 5.5 or greater and a second layer comprising an acid inhibitor, wherein at least a portion of the acid inhibitor is not surrounded by an enteric coating. In one embodiment, at least about 95%, at least about 99%, or at least about 99.5% of the acid inhibitor is not surrounded by an enteric coating. In yet another embodiment, the multilayer tablet is substantially free of sodium bicarbonate. In still another embodiment, the multilayer tablet is completely (i.e., 100%) free of sodium bicarbonate.

In one embodiment, the dosing regimen of the pharmaceutical compositions disclosed herein is one or more times daily. In another embodiment, the dosages are separated by a period of at least about 10 hours. In another embodiment, the pharmaceutical composition in unit dosage form is given before a patient ingests a meal, for example about 30-60 minutes prior to ingesting a meal. In another embodiment, the pharmaceutical compositions of the present disclosure may be administered therapeutically to patients either short term or over a longer period of time, for example chronically.

The pharmaceutical compositions disclosed herein include, but are not limited to, for example, tablets and capsules that can be made in accordance with methods that are standard in the art (see, e.g., Remington's Pharmaceutical Sciences, 16^{th} ed., A Oslo editor, Easton, Pa. (1980)). Suitable carriers include, but are not limited to: water; salt solutions; alcohols; gum arabic; vegetable oils; benzyl alcohols; polyethylene glycols; gelatin; carbohydrates such as lactose, amylose or starch; magnesium stearate; talc; silicic acid; paraffin; perfume oil; fatty acid esters; hydroxymethylcellulose; polyvinyl pyrrolidone; carnauba wax, colloidal silicon dioxide, croscarmellose sodium, glyceryl monostearate, hypromellose, methacrylic acid copolymer dispersion, methylparaben, polysorbate 80, polydextrose, povidone, propylene glycol, propylparaben, titanium dioxide, and triethyl citrate.

In one embodiment, at least one of the layers comprising the pharmaceutical compositions disclosed herein may be applied using standard coating techniques. The layer materials may be dissolved or dispersed in organic or aqueous solvents. The layer materials may include, but are not limited to, for example, one or more of the following materials: methacrylic acid copolymers, shellac, hydroxypropylmethcellulose phthalate, polyvinyl acetate phthalate, hydroxypropylmethyl-cellulose trimellitate, carboxymethyl-ethyl-cellulose, cellulose acetate phthalate, and/or other suitable polymer(s). The pH at which the first layer dissolves can be controlled by the polymer or combination of polymers selected and/or ratio of pendant groups. For example, dissolution characteristics of the polymer film can be altered by the ratio of free carboxyl groups to ester groups. The layers may also contain pharmaceutically acceptable plasticizers, such as, for example, triethyl citrate, dibutyl phthalate, triacetin, polyethylene glycols, polysorbates or other plasticizers. Additives may also be used in the pharmaceutical compositions disclosed herein, such as, for example, dispersants, colorants, anti-adhering, and anti-foaming agents.

In one embodiment, the pharmaceutical compositions disclosed herein can be in the form of a bi- or multi-layer tablet. In a bi-layer tablet, one portion/layer of the tablet contains the acid inhibitor, or a pharmaceutically acceptable salt thereof, in the required dosage along with any appropriate excipients, agents to aid dissolution, lubricants, fillers, and the like; and a second portion/layer of the tablet contains the aspirin or a pharmaceutically acceptable carrier thereof in the required dosage along with any excipients, dissolution agents, lubricants, fillers, and the like. In another embodiment, the aspirin or a pharmaceutically acceptable carrier portion/layer is surrounded by a polymeric coating that dissolves at a pH of at least about 3.5, 4.0, 4.5, 5.0, 5.5 or greater. In still another embodiment, the aspirin or a pharmaceutically acceptable carrier portion/layer is surrounded by a coating that delays release until the pH of the surrounding environment is at least about 3.5, 4.0, 4.5, 5.0, 5.5 or greater.

The aspirin, or a pharmaceutically acceptable salt thereof, may be granulated by methods such as slugging, low- or high- shear granulation, wet granulation, or fluidized-bed granulation. Of these processes, slugging generally produces tablets of less hardness and greater friability. Low-shear granulation, high-shear granulation, wet granulation and fluidized-bed granulation generally produce harder, less friable tablets.

### EXAMPLES

The invention is further defined in the following Examples. It should be understood the Examples are given by way of illustration only. From the above discussion and the Examples, one skilled in the art can ascertain the essential characteristics of the invention, and without departing from the spirit and scope thereof, can make various changes and modifications to adapt the invention to various uses and conditions. As a result, the invention is not limited by the illustrative Examples set forth herein, but rather defined by the claims appended hereto.

### Example 1

### Three Phase I, 4-Week Endoscopic Studies on PA32520 (Single-Tablet of EC-ASA 325 mg+IR Omeprazole 20 mg) and PA32540 (Single-Tablet of EC-ASA 325 mg+IR Omeprazole 40 mg), Showing a Decreased Risk of Gastroduodenal Mucosal Injury

A total of 240 healthy volunteers with normal baseline endoscopy (Lanza score 0) participated in three Phase I, single-blind, randomized, controlled studies to evaluate via endoscopy the gastroduodenal effects of a fixed combination tablet of delayed release (“DR”) aspirin (“ASA”) 325 mg and immediate release (“IR”) omeprozole (20 or 40 mg). Two studies evaluated PA32520 (DR ASA 325 mg +IR omeprazole 20 mg) vs. either EC-ASA 81 mg or 325 mg. The third study compared PA32540 (DR ASA 325 mg +IR omeprazole 40 mg) with EC-ASA 325 mg. All medications were dosed once daily for 4 weeks. Endoscopy results were evaluated using 1988 Lanza scoring, which is a system that scores the severity of NSAID-induced GI tract ulcers on a scale of 0=no visible lesions, 1=1 hemorrhage or erosion, 2=2-10 hemorrhages or erosions, 3=11-25 hemorrhages or erosions, 4=more than 25 hemorrhages or erosions or any ulcer. The primary endpoint was the proportion of subjects with Grade 3 or Grade 4 Lanza scores at week 4; additional assessments included incidence of gastric or duodenal ulcers (“GU/DU”) at 4 weeks and pharmacokinetics. Data were pooled across the 3 studies.

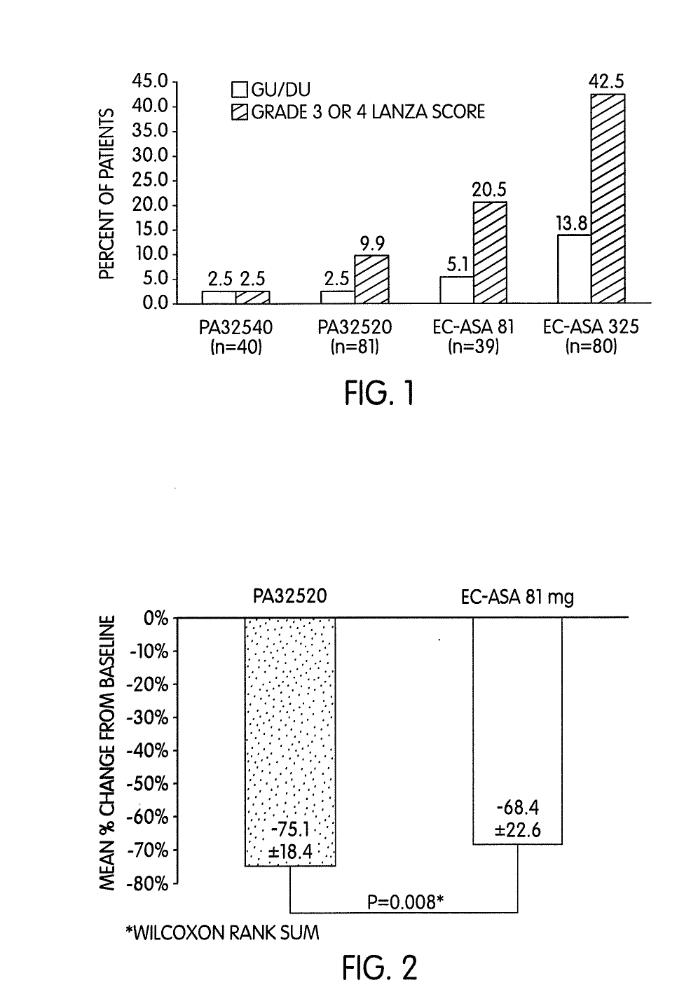

As shown in FIG. 1, Grade 3 or 4 Lanza scores and the incidences of GU/DU for the PA products were lower than for EC-ASA. With regard to Grade 3 or 4 Lanza scores, the results showed the following: PA32520 vs. EC-ASA 81 mg (9.9 vs. 20.5%, p=0.151); PA32520 vs. EC-ASA 325 mg (9.9% vs. 42.5%, p<0.001); PA32540 vs. EC-ASA 81 mg (2.5% vs. 20.5%, p=0.014); PA32540 vs. EC-ASA 325 mg (2.5% vs. 42.5%, p<0.001). With regard to the incidence of GU/DU, the results showed the following: PA32520 vs. EC-ASA 81 mg (2.5% vs. 5.1%, p=0.595); PA32520 vs. EC-ASA 325 mg (2.5% vs.13.8%, p=0.009); PA32540 vs. EC-ASA 81 mg (2.5% vs. 5.1%, p=0.615); PA32540 vs. EC-ASA 325 mg (2.5% vs. 13.8%, p=0.059). As shown in Table 1, Day 14 and Day 28 mean gastric pH values were higher with PA32520 than with EC-ASA, and a greater percent of PA32520 subjects had a pH of >3. Plasma salicylic acid pharmacokinetics were similar following dosing with PA32520 or PA32540 and EC-ASA 325 mg following both single-dose and repeat-dose administration. PA32520 was well tolerated and resulted in a similar frequency of GI adverse events as EC-ASA 325 mg. There was no statistically significant difference in gastroduodenal mucosal damage caused by 27 days of treatment with once daily PA32520 or EC-ASA 81 mg, although there was a trend to less damage with PA32520. PA32520 induced less GI mucosal damage than EC-ASA 81 mg based on Grade 3 or 4 Lanza scores for the duodenum at Day 14 and duodenal erosion counts at Day 14. PA32520 was statistically significantly better than EC-ASA aspirin 81 mg in increasing mean gastric pH at Day 14 and Day 28, and increasing the proportion of subjects with gastric pH >3 at Day 14.

TABLE 1

| | Day 14 | | | Day 28 | | |
|---|---|---|---|---|---|---|
| | PA32520 | EC ASA | | PA32520 | EC ASA | |
| | N = 40 (%) | N = 40 (%) | p-value¹ | N = 40 (%) | N = 40 (%) | p-value¹ |
| Mean (SD) | 4.2 (1.6) | 1.7 (0.6) | <0.001 | 3.5 (1.8) | 1.5 (0.4) | <0.001 |
| Median | 4.3 | 1.5 | | 2.8 | 1.5 | |
| Range | 1.5-6.0 | 1.0-3.5 | | 1.5-6.0 | 1.0-3.0 | |

¹Wilson Rank-Sum test
SD = standard deviation

Gastroduodenal Grade 3 or 4 Lanza scores and incidence of GU/DU for EC-ASA were dose-related. The fixed dose combination of DR ASA and IR omeprazole was associated with a significant reduction in gastroduodenal Grade 3 or 4 Lanza scores and GU/DU that were dose-related to the proton pump inhibitor. PA32540 demonstrated the least gastroduodenal damage and may provide an important option for at-risk patients who require long-term ASA therapy.

### Example 2

### Two Phase I, 4-Week Endoscopic Studies on PA32520 (Single-Tablet of EC-ASA 325 mg+IR Omeprazole 20 mg) Shows Greater Thromboxane Suppression and Lower Upper Gastrointestinal Damage

In a randomized, single-blinded controlled Phase I study, gastroduodenal mucosal changes using an established methodology (Lanza score) and urinary 11-dehydrothromboxane (“11-dh-TXB₂”) were determined in 80 healthy volunteers (mean ages 57-58 yrs) with no endoscopic evidence of gastroduodenal mucosal damage (Lanza score 0) who were treated with a daily dose of PA32520 or 81 mg EC-ASA. In a separate Phase I study (n=80), the effect of PA32520 vs. 325 mg EC-ASA alone on gastroduodenal mucosal changes was studied in 80 healthy volunteers. The primary endpoint was Lanza Grade 3 or 4 (>20 erosions/hemorrhages or ulcers) at Day 28; secondary endpoints included Grade 3 or 4 at Day 14, gastric or duodenal ulcers by Day 28, and the change from baseline in urinary 11-dh-TXB₂ after 4 weeks. Study assessments were conducted at baseline, Day 14, and Day 28.

As shown in Table 2, PA32520 was associated with 50%-84% less gastroduodenal mucosal damage than EC-ASA alone. As shown in FIG. 2, PA32520 was associated with a greater reduction in 11-dh-TXB₂ compared to EC-ASA 81 mg (−75% vs −68% mean percentage change from baseline, respectively; p=0.008). Over three times as many subjects in the PA32520 treatment group had reductions in urinary 11-dh-TXB₂ excretion rates from baseline to Day 27 in excess of 80% compared to the EC-ASA 81 mg treatment group.

TABLE 2

| | Study 1 | | | Study 2 | | |
|---|---|---|---|---|---|---|
| | PA32520 | EC-ASA 81 mg | | PA32520 | EC-ASA 325 mg | |
| Endpoint | N = 41 | N = 39 | p-Value | N = 40 | N = 40 | p-Value |
| Gastric and duodenal | 4 (9.8%) | 8 (20.5%) | 0.22 | 1 (2.5%) | 17 (42.5%) | <0.001 |
| Lanza 3 or 4 Scores | | | | | | |
| Day 14 | | | | | | |
| Gastric and duodenal | 4 (9.8%) | 8 (20.5%) | 0.22 | 3 (7.5%) | 19 (47.5%) | <0.001 |
| Lanza 3 or 4 Scores | | | | | | |
| Day 28* | | | | | | |

*primary analysis

Treatment with EC-ASA alone is associated with a high prevalence of UGI damage that is ameliorated by PA32520 therapy. Compared to EC-ASA 81 mg, PA32520 produces superior inhibition of in vivo thromboxane generation. PA32520 may provide an important option for at patients treated with ASA, as well as the great patient population that takes ASA intermittently, for short-term therapy, or chronically. High-dose ASA in combination with proton pump inhibitors may provide a reduction in UGI damage and greater thromboxane suppression.

### Example 3

### Four Phase 1, 4-Week Endoscopic Studies on PA32520 (Single-Tablet of EC-ASA 325 mg+IR Omeprazole 20 mg) and PA32540 (Single-Tablet of EC-ASA 325 mg+IR Omeprazole 40 mg) Show Bioequivalence to EC-ASA, Greater Thromboxane Suppression and Lower Upper Gastrointestinal Damage

Four Phase I studies with PA32520 and PA32540 evaluated bioequivalence to EC-ASA, UGI safety, and inhibition of thromboxane. The bioequivalence of aspirin from PA32540 vs. EC-ASA 325 mg/day was determined in a single-dose, open-label, crossover study in 36 healthy volunteers (mean age 32 yrs). In three single-blind, multiple-dose, randomized studies, healthy adults >50 yrs with normal baseline endoscopy (Lanza score 0) were treated with either PA32520, PA32540, EC-ASA 81 mg/day or EC-ASA 325 mg/day. For PA32520 vs. EC-ASA 81 mg/day, 11-dh-TXB₂ was also measured. The endpoints were the proportion of subjects with Grade 3 or 4 Lanza scores at Day 14, the proportion of subjects with Grade 3 or 4 Lanza scores at Day 28, and the concentration of urinary 11-d-TXB₂ after 4 weeks of therapy.

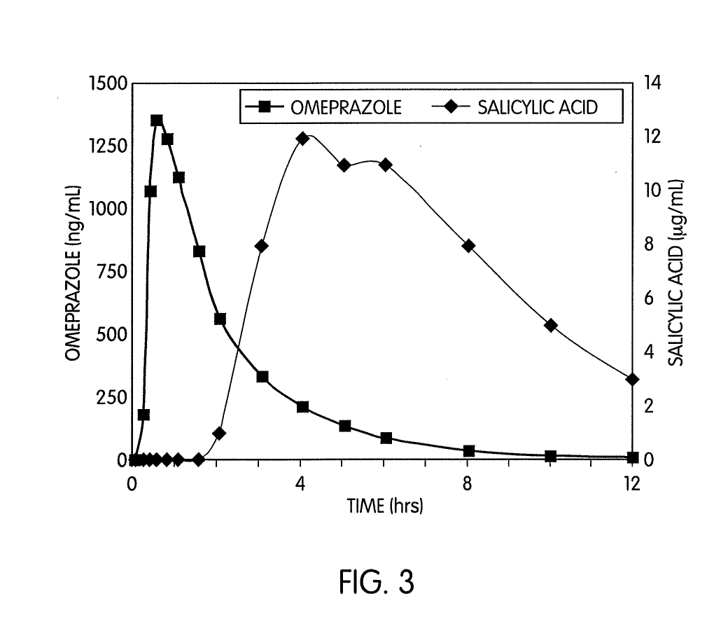

PA32540 was found to be bioequivalent to EC-ASA 325 mg/day; the geometric LSM ratio (90% CI) for AUC_{0-infinity} was 1.095 (0.967, 1.239) and for Cₘₐₓ was 1.077 (0.959, 1.209). FIG. 3 shows the release profile of PA32540 at Day 13; IR omeprazole in PA32540 has no effect on the pharmacokinetic profile of salicylic acid. Omeprazole was rapidly absorbed from PA32540 and eliminated from the systematic circulation with a mean elimination half life of approximately 1 hour. Plasma exposure of salicylic acid from PA32540 was similar to marketed EC-ASA 325 mg following both single-dose and repeat-dose administration of PA32540. This observation rules out lower dosage aspirin systematic exposure as the explanation for the reduction in damage associated with PA32540. Additionally, it shows that immediate release omeprazole in PA32540 has no effect on salicylic acid pharmacokinetics. Chronic administration of PA32540 was well tolerated. After 4 weeks of therapy, PA was associated with an 84%-90% reduction in UGI injury (Lanza score 3 or 4, >20 erosions, hemorrhages, or ulcers) compared with EC-ASA 325 mg/day (p <0.003). Lanza score 3 or 4 level injury at Day 28 occurred in 9.8% of PA32520 patients and in 20.5% of EC-ASA 81 mg/day patients (p=0.22). Urinary 11-dh-TXB₂ at baseline was 853.2 pg/mg creatinine (“Cr”) for PA32520 and 884.6 pg/mg Cr for EC-ASA 81 mg/day (p=0.97). As shown in Table 3, after 4 weeks of treatment, 11-dh-TXB₂ was significantly lower for PA32520 (175.5 pg/mg Cr) than for EC-ASA 81 mg/day (245.2 pg/mg Cr); p=0.005.

TABLE 3
URINARY 11-D-TXB2 AFTER 4 WEEKS OF TREATMENT

| Urinary 11-d-TXB2 | | |
|---|---|---|
| (pg/mg Cr) | PA 32520 (n = 41) | EC-ASA 81 mg (n = 39) |
| Minimum | 48.7 | 48.2 |
| First Quartile | 132.6 | 181.4 |
| Median | 188.1 | 258.4 |
| Third Quartile | 233.6 | 327.8 |
| Maximum | 852.2 | 679.5 |
| Geometric Mean | 175.5* | 245.2 |

*P = 0.005

PA32540 is bioequivalent to EC-ASA 325 mg/day, but with a significant improvement in UGI safety. Also, PA32520 inhibits urinary 11-dh-TXB₂ significantly more than EC-ASA 81 mg/day. PA was associated with a significant reduction in gastroduodenal injury, and PA32540 demonstrated the least gastroduodenal damage and fewest overall GI adverse events. Thus, while secondary prevention of strokes and transient ischemic attacks with ASA alone is associated with UGI damage and as such may require lower doses of ASA or alternative anti-thrombotic agents, PA may allow for higher doses of ASA, for example for secondary prevention of cardiovascular disease, strokes and transient ischemic attacks.

### Example 4

### Phase 1, 4-Week Endoscopic Study on PA65020 (Two Tablets of EC-ASA 325 mg+IR Omeprazole 20 mg) at Analgesic Doses that Shows Significant Reduction of Incidence of Gastroduodenal Ulcers

In a single-center, Phase 1, randomized, double-blind study, PA65020 (n=20) or EC-ASA 650 mg (n=20) was administered in the clinic twice daily for 28 days to healthy volunteers (≧50 yrs) with normal baseline endoscopy (Lanza score 0). Each dose of PA65020 was administered as one tablet of PA32520 and one tablet of EC-ASA 325 mg. EC-ASA 650 mg was administered as two EC-ASA 325 mg tablets. The total daily ASA dose was 1300 mg. Outcome evaluations included the occurrence of endoscopically proven gastric and/or duodenal lesions meeting Grade 3 or Grade 4 Lanza scores on Day 28 (primary endpoint), incidence of gastroduodenal ulcers, as well as assessments of dyspepsia-associated abdominal pain by mSODA (modified severity of dyspepsia assessment score, range 2-47), heartburn, and adverse events.

A total of 40 subjects (mean age 59.7 years) were treated. As shown in Table 4, at Day 28, the incidence of Grade 3 or 4 Lanza scores was significantly less for the PA65020 group (3, or 15%) than for the EC-ASA 650 mg group (17, or 85%), P<0.001. The incidence of GU/DU on Day 28 was also significantly lower with PA65020 vs. EC-ASA 650 mg (0% vs. 40%, P=0.003). At Day 28, the mean change from baseline in mSODA was 0 for PA65020 and 0.7 for EC-ASA 650 mg. More PA65020 subjects were heartburn-free (90%) throughout the study compared with subjects in the EC-ASA 650 mg group (75%). Mean salicylic acid trough levels were similar between PA65020 and EC-ASA 650 treatment groups on both Day 14 (17.8 mcg/mL vs. 19.0 mc/mL) and Day 28 (13.5 mcg/mL v. 13.3 mcg/mL), so the differences in salicylic acid levels cannot explain the reduction in Lanza scores of the absence of ulcers in the PA65020 treatment as compared to the EC-ASA 650 mg treatment. The most commonly reported adverse events were GI-related, primarily dyspepsia (2 subjects in each treatment group) and stomach discomfort (3 subjects in the EC-ASA 650 mg group vs. 0 subjects in the PA65020 group).

TABLE 4

| | PA65020 | EC-ASA 650 mg | |
|---|---|---|---|
| | N = 20 | N = 20 | |
| Endpoint | n (%) | n (%) | P-value |
| Gastric and duodenal | | | |
| Lanza 3 or 4 scores | | | |
| Day 14 | 1 (5%) | 18 (90%) | <0.001 |
| Day 28 | 3 (15%) | 17 (85%) | <0.001 |
| GU/DU | | | |
| Day 14 | 0 | 4 (20%) | 0.106 |
| Day 28 | 0 | 8 (40%) | 0.003 |

Analgesic doses of over-the-counter ASA produced significant mucosal damage in most subjects following 1 month of treatment. PA65020 is associated with a significantly decreased risk of GU/DU, and may provide an important option for at-risk patients who require analgesic doses of ASA.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of treating a patient at risk of developing an NSAID-associated ulcer for a disease or disorder that responds to aspirin, comprising administering to said patient a pharmaceutical composition in unit dosage form comprising
a) omeprazole or pharmaceutically acceptable salt thereof, that is immediately soluble when the dosage form is placed in an aqueous medium, independent of pH, in an amount effective to raise the gastric pH of the patient to at least 3.5 upon administration of one or more of the unit dosage forms, and
b) aspirin or a pharmaceutically acceptable salt thereof, wherein the aspirin or a pharmaceutically acceptable salt thereof is surrounded by a coating that is substantially insoluble in an aqueous medium at a pH below 3.5 and at a temperature of 37° C.;
wherein said administration is continued for a period of at least 14 days.

2. The method of claim 1, wherein said patient is administered one or more of said unit dosage forms daily for a period of at least 28 days.

3. The method of claim 1, wherein said patient is at increased risk of ulcer formation due to said patient's age.

4. The method of claim 1, wherein the omeprazole or a pharmaceutically acceptable salt thereof, is present in an amount effective to raise the pH of the gastric fluid of the patient to at least 4.5 when the dosage form is administered orally.

5. The method of claim 1, wherein the amount of aspirin, or a pharmaceutically acceptable salt thereof, is present in said unit dosage form at 81-650 mg.

6. The method of claim 1, wherein the amount of aspirin, or a pharmaceutically acceptable salt thereof, is present in said unit dosage form at 325-650 mg.

7. The method of claim 1, wherein the amount of omeprazole, or a pharmaceutically acceptable salt thereof, is present in said unit dosage form at 15-40 mg.

8. The method of claim 7, wherein the amount of aspirin, or a pharmaceutically acceptable salt thereof, is present in said unit dosage form at 81-650 mg

9. The method of claim 1, wherein said patient is treated for pain or inflammation.

10. The method of claim 9, wherein said pam or inflammation is associated with osteoarthritis; rheumatoid arthritis; ankylosing spondylitis; headache; toothache; common cold; muscle ache; cardiovascular disease; cancer; cerebrovascular disease; or a combination thereof.

11. The method of claim 1, wherein the pharmaceutical composition reduces heartburn or dyspepsia associated symptoms in said patient.

12. The method of claim 1, wherein the unit dosage form is a tablet comprising a core and two or more layers, in which:
a) the core comprises aspirin or a pharmaceutically acceptable salt thereof;
b) a first layer surrounds the core and has a coating substantially insoluble in aqueous medium at a pH below 3.5; and
c) at least one second layer comprising the omeprazole, or a pharmaceutically acceptable salt thereof, said second layer surrounding the coating of said first layer.

13. The method of claim 12, wherein the amount of omeprazole, or a pharmaceutically acceptable salt thereof, is present in said unit dosage form at 15-40 mg and the amount of aspirin, or a pharmaceutically acceptable salt thereof, is present in said unit dosage form at 81-650 mg.

14. The method of claim 13, wherein said patient is treated for pain, or inflammation.

15. The method of claim 14, wherein the unit dosage form provides for the coordinated release of omeprazole, or a pharmaceutically acceptable salt thereof, and the aspirin or a pharmaceutically acceptable salt thereof.

16. The method of claim 2, wherein:
a) the amount of omeprazole, or a pharmaceutically acceptable salt thereof, is present in said unit dosage form at 15-40 mg; and
b) the amount of aspirin, or a pharmaceutically acceptable salt thereof, is present in said unit dosage form at 81-650 mg.

17. The method of claim 16, wherein said patient is treated for pain or inflammation.

18. The method of claim 17, wherein said pain or inflammation is associated with osteoarthritis; rheumatoid arthritis; ankylosing spondylitis; headache; toothache; common cold; muscle ache; cardiovascular disease; cancer; cerebrovascular disease; or a combination thereof.

19. The method of claim 18, wherein the unit dosage form is a tablet comprising a core and two or more layers, in which:
a) the core comprises aspirin or a pharmaceutically acceptable salt thereof;
b) a first layer surrounds the core and is a coating substantially insoluble in aqueous medium at a pH below 3.5; and
c) at least one second layer comprising the omeprazole or pharmaceutically acceptable salt thereof surrounds the coating of the first layer.

20. The method of claim 19, wherein the pharmaceutical composition reduces heartburn or dyspepsia associated symptoms in said patient.
